(19) United States
(12) Patent Application Publication
(10) Pub. No.: US 2014/0370098 A1
(43) Pub. Date: Dec. 18, 2014

(54) TINTED EMULSION

(71) Applicant: L'OREAL, PARIS (FR)

(72) Inventors: Isabelle Terrisse, Vitry Sur Seine (FR); Charlotte Lemaire, Antony (FR)

(21) Appl. No.: 14/360,784

(22) PCT Filed: Nov. 23, 2012
(86) PCT No.: PCT/IB2012/056660

§ 371 Date: May 27, 2014

Related U.S. Application Data
(60) Provisional application No. 61/567,661, filed on Dec. 7, 2011.

(30) Foreign Application Priority Data
Nov. 25, 2011 (FR) 1160827

(51) Int. Cl. A61K 8/06; A61Q 1/02; A61K 8/37; A61K 8/73; A61K 8/60; A61K 8/19; A61K 8/29

(52) U.S. Cl. CPC A61K 8/062; A61K 8/19; A61Q 1/02; A61K 8/29; A61K 8/73; A61K 8/602; A61K 8/37; A61K 2800/413; A61K 2800/43; A61K 2800/591
USPC 424/489; 424/63

(57) ABSTRACT

The present invention relates to a cosmetic composition for caring for and/or making up keratin materials which is in the form of an oil-in-water emulsion which undergoes phase inversion when it is applied to said keratin materials, comprising: (i) at least one pigment having a particle size greater than 100 nanometers, and (ii) a combination of at least one hydrophilic surfactant and at least one lipophilic surfactant. It also relates to the associated cosmetic process.

The subject of the invention is a cosmetic composition comprising pigments, which has the capacity to undergo phase inversion at the time of application to keratin materials.

For reasons of homogeneous pigment distribution, the cosmetic formulas using pigments are most commonly in the form of a water-in-oil emulsion. These compositions have, moreover, the advantage of conferring good coverage on the skin. In these compositions, the pigments solubilized in the oily phase are distributed better over the skin and thus the pigments are in the external phase, compared with emulsions of oil-in-water emulsion type, where the pigments are in the internal phase.

It so happens that, for a few years, textures conferring properties of freshness on application and giving a lighter sensoriality than that conferred by water-in-oil emulsions have been more particularly sought in the cosmetics industry. Consequently, compositions of oil-in-water (O/W) type, consisting of an aqueous dispersant continuous phase and an oily dispersed discontinuous phase, are particularly sought in the cosmetics industry owing to the fresher, less greasy and lighter feel than when water-in-oil (W/O) emulsions are applied.

It is also noted that, in the particular case of compositions containing natural ingredients or ingredients of natural origin, namely not comprising silicone ingredients in particular, the sensation of greasiness and heaviness can be accentuated for water-in-oil emulsions.

The present invention therefore aims to provide compositions containing pigments, which have properties on application similar to those of oil-in-water emulsions, while at the same time providing uniform pigment distribution and good coverage on the skin, like the properties of water-in-oil emulsions.

Oil-in-water emulsions which invert to give water-in-oil emulsions at the time of application to the skin are known from documents EP 1 917 954 and WO 2009/043533 in the field of suntan products.

The technology implemented in these two phase-inversion patent applications is also called SWOP or SWITCH WATER OIL PHASE.

These documents make no reference to cosmetic products of tinted composition type.

The applicant has discovered that the combination of at least one hydrophilic surfactant and at least one lipophilic surfactant, used with a pigment having at least a particle size greater than 100 nm and preferably a polysaccharide derivative, makes it possible to obtain an oil-in-water emulsion composition which undergoes phase inversion when it is applied in particular to the skin and which has good cosmetic properties, in particular both a sensation of freshness on application and an appropriate coverage.

Thus, the present invention relates to a cosmetic composition for caring for and/or making up keratin materials which is in the form of an oil-in-water emulsion which undergoes phase inversion when it is applied to said keratin materials, comprising:
(i) at least one pigment having a particle size greater than 100 nanometers, and
(ii) a combination of at least one hydrophilic surfactant and at least one lipophilic surfactant.

The term <<natural compound>> means a compound which is obtained directly from the earth or the soil or from plants or animals, via, as appropriate, one or more physical processes, such as, for example, milling, refining, distillation, purification or filtration.

The term “compounds of natural origin” means a natural compound that has undergone one or more additional chemical or industrial treatments, giving rise to modifications that do not affect the essential qualities of this compound and/or a compound predominantly comprising natural constituents that may or may not have undergone transformations as indicated above.

Mention may be made, as nonlimiting example of additional chemical or industrial treatment bringing about modifications which do not affect the essential qualities of a natural compound, of those allowed by the controlling bodies, such as Ecocert (Reference system for biological and ecological cosmetic products, January 2003), or defined in recognized handbooks in the field, such as Cosmetics and Toiletries Magazine, 2005, Vol. 120, 9:10.

The composition according to the invention is intended for topical application and thus comprises a physiologically acceptable medium. “Physiologically acceptable medium” is understood here to mean a medium compatible with keratin materials, such as the skin, mucous membranes, scalp or eyes and/or keratin fibers, such as the eyelashes or hair.

The present invention also relates to a cosmetic treatment process for keratin materials, characterized in that a composition in accordance with the present invention is applied to said keratin materials.

Phase Inversion Technology

The phase inversion technology used in the present invention is, as indicated above, the SWOP technology: the oil-in-water emulsion composition undergoes phase inversion during its application owing to the friction caused, in other words by virtue of the mechanical energy generated by the movements made by the user on application.

In fact, by means of this technique, a lipophilic film is obtained on the skin that is faster than the inversion process obtained conventionally when an oil-in-water emulsion is applied and the aqueous phase evaporates.

In sensory terms, in particular from the point of view of the absorption, the tacky aspect and the softness of the application, the advantages of oil-in-water emulsions are observed while at the same time the advantages of water-in-oil emulsions are retained. It is the choice of one or more surfactant(s) that is determining in providing this phase inversion, as it is detailed hereinafter.

Pigments

A composition according to the invention comprises at least one pigment having a particle size greater than 100 nm. In particular, the particle size can be greater than 300 nm, and even more preferentially greater than 400 nm.

The pigments having a particle size greater than 100 nm can be present in a proportion of from 0.1 to 40% by weight, preferably from 1 to 30% by weight, or even from 5 to 30% by weight, relative to the total weight of the composition containing them.

The term “pigments” should be understood as meaning white or colored, mineral or organic particles that are insoluble in an aqueous solution, which are intended to color and/or opacify the composition containing them.

The term “particle size” means, in the context of the present invention, the size of the particles or aggregates of particles. In particular, it is the average size of the particles or aggregates of particles.

The average size of the particles corresponds to the volume-average diameter (D50) measured by laser diffraction particle size analysis or other equivalent method known to those skilled in the art.

The pigments may be white or colored, and mineral and/or organic.

As mineral pigments that may be used in the invention, mention may be made of titanium oxide, titanium dioxide, zirconium oxide, zirconium dioxide, cerium oxide or cerium dioxide and also zinc oxide, iron oxide or chromium oxide, ferric blue, manganese violet, ultramarine blue and chromium hydrate, and mixtures thereof.

It may also be a pigment having a structure that may be, for example, of sericite/brown iron oxide/titanium dioxide/silica type. Such a pigment is sold, for example, under the reference COVERLEAF NS or JS by the company CHEMICALS AND CATALYSTS.

They may also be pigments having a structure that may be, for example, of silica microsphere type containing iron oxide. An example of a pigment having this structure is the product sold by the company MIYOSHI under the reference PC BALL PC-LL-100 P, this pigment consisting of silica microspheres containing yellow iron oxide.

Advantageously, the pigments in accordance with the invention are iron oxides and/or titanium dioxides.

They can in particular be chosen from the iron oxides (and) disodium stearoyl glutamate (and) aluminum hydroxide sold by the company Myoshi Kase under the name NAI-C33-7001-10, NAI-C33-8001-10 or NAI-C33-9001-10 or else the titanium dioxide (and) disodium stearoyl glutamate (and) aluminum hydroxide sold by the company Myoshi Kase under the name NAI-TAO-77891.

Provided that their presence does not affect the expected properties, the cosmetic composition of the present invention can also contain, in addition, pigments having a particle size of less than 100 nm.

Combination of at Least One Hydrophilic Surfactant and at Least One Lipophilic Surfactant

For the purpose of the present invention, the hydrophilic or lipophilic nature of the surfactant is evaluated from the viewpoint of the HLB value.

The GRIFFIN HLB value is defined in J. Soc. Cosm. Chem. 1954 (volume 5), pages 249-256.

Reference may also be made to the document “Encyclopedia of Chemical Technology, KIRK-OTHMER”, volume 22, pp. 333-432, 3rd edition, 1979, WILEY, for the definition of the properties and (emulsifying) functions of surfactants, in particular pp. 347-377 of this reference, for anionic, amphoteric and nonionic surfactants.

Hydrophilic Surfactant

A composition according to the invention can comprise from 0.2 to 2% by weight, and preferably from 0.5 to 1% by weight, of hydrophilic surfactants, relative to the total weight of the composition.

For the purposes of the present invention, a hydrophilic surfactant is understood to be a surfactant having an HLB greater than or equal to 13.

By way of nonlimiting illustration of hydrophilic surfactants in accordance with the present invention, mention may in particular be made of the following surfactants:
Alkyl mono- and polyglucosides:

The alkyl polyglucoside(s) is (are) well known and can be more particularly represented by the following general formula:

R₉O—(R₁₀O)_{t′}-(G)ᵥ(I)

in which R₉ represents a linear or branched alkyl and/or alkylene radical containing approximately from 12 to 30 carbon atoms, or an alkylphenyl radical of which the linear or branched alkyl radical contains from 12 to 30 carbon atoms, R₁₀ represents one or more alkylene radicals containing approximately from 2 to 4 carbon atoms, G represents a sugar unit containing from 5 to 6 carbon atoms, t′ denotes a value ranging from 0 to 10, preferably 0 to 4, and v denotes a value ranging from 1 to 15.

Alkyl polyglucosides which are preferred according to the present invention are compounds of formula (I) in which R₉ denotes more particularly a linear or branched, saturated or unsaturated alkyl radical containing from 12 to 30 carbon atoms, t′ denotes a value ranging from 0 to 3 and even more particularly equal to 0, G can denote glucose, fructose or galactose, preferably glucose. The degree of polymerization, i.e. the value of v in formula (I), can range from 1 to 15, preferably from 1 to 4. The average degree of polymerization can be more particularly between 1 and 2 and even more particularly from 1.1 to 1.5. The glycosidic linkages between the sugar units are of 1-6 or 1-4 and preferably 1-4 type.

Compounds of formula (I) are in particular represented by the products sold by the company COGNIS under the name PLANTAREN® (600 CS/U, 1200 and 1300) or PLANTACARE® (818 and 1200);
Anionic derivatives of alkyl polyglucosides:

The anionic derivatives of alkyl polyglucosides can in particular be citrates, tartrates, sulfonates, carboxylates, carbonates and ethers of glycerol obtained from alkyl polyglucosides. Mention may be made, for example, of the sodium salt of cocoyl polyglucoside (1,4) tartaric ester, sold under the name EUCAROL AGE-ET® by the company Cesalpinia, the disodium salt of cocoyl polyglucoside (1,4) sulfosuccinic ester, sold under the name ESSAI 512 MP® by the company SEPPIC, the sodium salt of cocoyl polyglucoside (1,4) citric ester, sold under the name EUCAROL AGE-EC® by the company Cesalpinia, or the sodium salt of sulfonate ester of a mixture of hydroxypropyl laurylmonoglucoside and of hydroxypropyl lauryldiglucoside (lauryl glucoside hydroxypropylsulfonate), sold under the name SUGANATE 160 by the company COLONIAL CHEMICAL.

According to one particular embodiment, it is an alkyl monoglucoside and even more particularly an alkyl monoglucose. In particular, mention may be made of lauryl glucoside.

According to another particular embodiment, it is an anionic derivative of an alkyl monoglucoside. In particular, the alkyl monoglucoside derivative can be carboxymethylated. In this respect, mention may be made of sodium carboxymethyl ether of lauryl glucoside.

According to yet another particular embodiment of the invention, the hydrophilic surfactant comprises at least one alkyl monoglycoside, such as an alkyl monoglucose or alkyl monoglucoside, and an anionic derivative of an alkyl monoglucoside such as an alkyl monoglucoside carboxylate, which is in particular carboxymethylated.

In this respect, mention may in particular be made of sodium lauryl glucose carboxylate (and) lauryl glucoside, otherwise known as sodium carboxymethyl ether of lauryl glucoside, in particular as sold by COGNIS under the name Plantapon® LGC Sorb;
fatty acid esters, in particular of glycerol:

mention may in particular be made of polyglyceryl-6 caprylate, in particular as sold by TAIYO KAGAKU under the name Sunsoft Q-81F;

mention may in particular be made of polyglyceryl-10 oleate, in particular as sold by TAIYO KAGAKU under the name Sunsoft Q-171S;

mention may in particular be made of polyglyceryl-5 laurate, in particular as sold by STRAETMANS under the name Dermo feel G5 L or else by TAIYO KAGAKU under the name Sunsoft A-12E;

mention may in particular be made of polyglyceryl-4 caprate, in particular as sold by EVONIK GOLDSCHMIDT under the name Tegosoft PC 41;

mention may in particular be made of polyglyceryl-10 myristate, in particular as sold by TAIYO KAGAKU under the name Sunsoft Q-14S;

mention may in particular be made of polyglyceryl-10 stearate, in particular as sold by TAIYO KAGAKU under the name Sunsoft Q-18Y;

mention may in particular be made of polyglyceryl-5 stearate, in particular as sold by TAIYO KAGAKU under the name Sunsoft A-18E;

mention may in particular be made of polyglyceryl-10 laurate, in particular as sold by STRAETMANS under the name Dermofeel G10 L or else by TAIYO KAGAKU under the name Sunsoft Q-12S;

mention may in particular be made of polyglyceryl-5 myristate, in particular as sold by TAIYO KAGAKU under the name Sunsoft A-14E;

mention may in particular be made of polyglyceryl-5 laurate, in particular as sold by TAIYO KAGAKU under the name Sunsoft A-12E;
polysorbates:

mention may in particular be made of polysorbate 21, in particular as sold by CRODA under the name Tween 21;

mention may in particular be made of polysorbate 60, in particular as sold by CRODA under the name Tween 80 V;

mention may in particular be made of polysorbate 80, in particular as sold by CRODA under the name Crillet 4 super;

mention may in particular be made of polysorbate 40, in particular as sold by CRODA under the name Tween 40;
sucrose esters:

mention may in particular be made of sucrose stearate, in particular as sold by CRODA under the name Crodesta F-160 or sold by SISTERNA under the name Sisterna SP70-C or else sold by MITSUBISHI KAGAKU FOOD under the name Ryoto sugar ester S1570;

mention may in particular be made of sucrose laurate, in particular as sold by MITSUBISHI KAGAKU FOOD under the name Surfhope SE COSME C-1216;

mention may in particular be made of sucrose palmitate, in particular as sold by MITSUBISHI KAGAKU FOOD under the name Surfhope SE COSME C-1616;

mention may in particular be made of sucrose myristate, in particular as sold by MITSUBISHI KAGAKU FOOD under the name Surfhope SE COSME C-1416;

mention may in particular be made of sucrose cocoate, in particular as sold by EVONIK under the name TEGOSOFT LSE 65 K SOFT;
other hydrophilic surfactants with an HLB greater than or equal to 13 may also be mentioned:

laureth-4 phosphate, in particular as sold by CLARIANT under the name Hostaphat KL 340 D;

lauroyl sarcosine, in particular as sold by CRODA under the name Crodasinic L;

glycereth-25 PCA isostearate, in particular as sold by NIHON EMULSION under the name Pyroter GPI-25.

Lipophilic Surfactants

A composition according to the invention may comprise from 2% to 7% by weight and preferably from 3% to 5% by weight of lipophilic surfactants, relative to the total weight of the composition.

For the purposes of the invention, a lipophilic surfactant is understood to be a surfactant having an HLB less than or equal to 9.

By way of nonlimiting illustration of lipophilic surfactants in accordance with the present invention, mention may in particular be made of the following surfactants:
fatty acid esters, in particular of glycerol and of polyglycerol.

According to one particular embodiment of the invention, the fatty acid ester(s) of polyglycerol is (are) chosen from esters resulting from the reaction of polyglycerol comprising from 2 to 12 glycerol units, preferably from 3 to 10 glycerol units, and of at least one fatty acid containing from 8 to 24 carbon atoms, preferably from 8 to 22 carbon atoms, better still from 10 to 20 carbon atoms and even better still from 10 to 18 carbon atoms. The fatty acids containing from 8 to 24 carbon atoms may be linear or branched, and saturated or unsaturated.

The fatty acids may be chosen from oleic acid, stearic acid, isostearic acid, lauric acid, palmitic acid, myristic acid, linoleic acid, capric acid and caprylic acid, or mixtures thereof.

The fatty acid esters of polyglycerol can be chosen from monoesters, diesters, triesters and tetraesters, polyesters and mixtures thereof. Use is preferably made of esters with a low degree of esterification, for instance fatty acid monoesters, diesters or triesters of polyglycerol, or a mixture. The fatty acid ester of polyglycerol can be in the form of a mixture of esters with a low degree of esterification, for instance a mixture of monoester and diester or a mixture of monoester, diester and triester.

According to one embodiment, the fatty acid ester of polyglycerol is chosen from esters resulting from the reaction of polyglycerol comprising from 3 to 10 glycerol units and of at least one fatty acid containing from 8 to 20 carbon atoms, preferably from 10 to 18 carbon atoms, such as stearic acid or isostearic acid.

Mention may in particular be made of polyglyceryl-2 distearate, in particular as sold by NIHON EMULSION under the name Emalex PGSA;

mention may in particular be made of polyglyceryl-10 decastearate, in particular as sold by TAIYO KAGAKU under the name Sunsoft Q-1810S;

mention may in particular be made of glyceryl oleate, in particular as sold by COGNIS under the name Monomuls 90-O 18;

mention may in particular be made of glyceryl stearate, in particular as sold by COGNIS under the name Cutina GMS V;

mention may in particular be made of polyglyceryl-5 hexastearate, in particular as sold by TAIYO KAGAKU under the name Sunsoft A-186E;

mention may in particular be made of polyglyceryl-10 pentaoleate, in particular as sold by TAIYO KAGAKU under the name Sunsoft Q-175S;

mention may in particular be made of polyglyceryl-10 pentastearate, in particular as sold by TAIYO KAGAKU under the name Sunsoft Q-185S;

mention may in particular be made of glyceryl caprylate/caprate, in particular as sold by STEPAN under the name Stepan Mild GCC;

mention may in particular be made of polyglyceryl-10 heptaoleate, in particular as sold by TAIYO KAGAKU under the name Sunsoft Q-177S;

mention may in particular be made of polyglyceryl-4 isostearate, in particular as sold by EVONIK GOLDSCHMIDT under the name Isolan GI 34;

mention may in particular be made of diisostearoyl polyglyceryl-3 dimer dilinoleate, in particular as sold by EVONIK GOLDSCHMIDT under the name Isolan PDI;

mention may in particular be made of glyceryl laurate, in particular as sold by COGNIS under the name Monomuls 90-L 12;

mention may in particular be made of polyglyceryl-5 trioleate, in particular as sold by TAIYO KAGAKU under the name Sunsoft A-173E;

mention may in particular be made of polyglyceryl-2 oleate, in particular as sold by TAIYO KAGAKU under the name Sunsoft Q-17B;

mention may in particular be made of polyglyceryl-5 trimyristate, in particular as sold by TAIYO KAGAKU under the name Sunsoft A-143E;

mention may in particular be made of polyglyceryl-2 caprylate, in particular as sold by TAIYO KAGAKU under the name Sunsoft Q-81B;

mention may in particular be made of polyglyceryl-2 laurate, in particular as sold by TAIYO KAGAKU under the name Sunsoft Q-12D.

According to one particular embodiment, the fatty acid which is suitable for the reaction with the ester(s) of polyglycerol comprises at least one hydroxyl group. This is the case for ricinoleic acid.

Mention may in particular be made of polyglyceryl-3 ricinoleate (and) sorbitan isostearate, in particular as sold by CRODA under the name Arlacel 1690.

Mention may in particular be made of polyglyceryl-3 ricinoleate, in particular as sold by AARHUSKARLSHAMN under the name Akoline PGPR.

According to another particular embodiment, the fatty acid which is suitable for the reaction with the ester(s) of polyglycerol is a polyacid comprising at least one hydroxyl group.

Thus, according to one variant of the invention, the hydrophobic surfactant comprises at least one polyglyceryl polyricinoleate.

For the purpose of the invention, the term “polyglyceryl polyricinoleate” denotes an ester resulting from the esterification of one or more polyglycerols with at least one polyricinoleic acid.

A polyglycerol which is suitable for the invention can be chosen from the compounds of general formula (II) below:

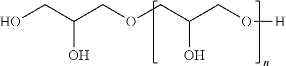

in which n represents an integer between 1 and 11, and in particular between 1 and 7, preferably between 1 and 5, even more particularly between 1 and 2.

A polyricinoleic acid which is suitable for the invention can be chosen from the compounds of general formula (III) below:

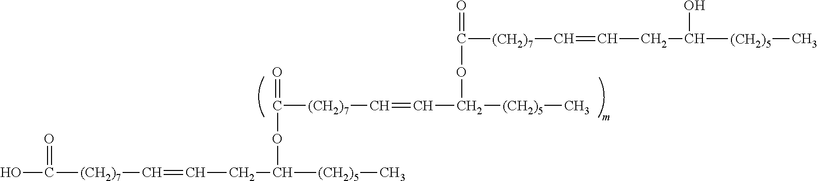

in which m represents an integer between 0 and 10, in particular between 1 and 8, and more particularly between 1 and 5.

A polyglyceryl polyricinoleate which is suitable for the invention can be a total or partial ester.

Preferably, a polyglyceryl polyricinoleate which is suitable for the invention is a partial ester.

For the purpose of the invention, the term “partial ester” is intended to denote a compound in which not all the —OH groups of the polyglycerol units have been esterified with polyricinoleic acid, in other words a polyglyceryl polyricinoleate compound comprising at least one free—OH group on the polyglycerolated chain.

By way of example, a polyglyceryl polyricinoleate which is suitable for the invention can be a compound of general formula (IV) below:

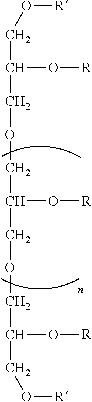

in which R and R′ represent, independently, radicals chosen from a hydrogen atom or a polyricinoleate chain, with the proviso that at least one of these R or R′ radicals is a polyricinoleate chain.

Preferably, at least one of the R or R′ groups of the polyglycerolated chain is a hydrogen atom.

According to one particular embodiment of the invention, the polyglyceryl polyricinoleate(s) present in the composition is (are) chosen from polyglyceryl-3 polyricinoleate and polyglyceryl-6 polyricinoleate, and mixtures thereof.

Mention may in particular be made of polyglyceryl-6 polyricinoleate, in particular as sold by NIKKOL under the name Hexaglyn PR-15;

Mention may in particular be made of polyglyceryl-3 polyricinoleate, in particular as sold by CRODA under the name Crester PR.

According to yet another particular embodiment, the fatty acid which is suitable for the reaction with the ester(s) of polyglycerol comprising at least one hydroxyl group is polyhydroxystearic acid.

Mention may in particular be made of polyglyceryl-4 diisostearate polyhydroxystearate sebacate, in particular as sold by EVONIK GOLDSCHMIDT under the name Isolan GPS;

Mention may in particular be made of polyglyceryl-2 dipolyhydroxystearate, in particular as sold by COGNIS under the name DEHYMULS DPGP;

Mention may in particular be made of polyhydroxystearic acid diesters, in particular polyglyceryl-2 dipolyhydroxystearate, in particular as sold by COGNIS under the name Dehymuls PGPH.
polysorbates:

mention may in particular be made of sorbitan tristearate, in particular as sold by CRODA under the name Span 65;

mention may in particular be made of sorbitan sesquioleate, in particular as sold by CRODA under the name Arlacel 83 V;

mention may in particular be made of sorbitan isostearate, in particular as sold by CRODA under the name Arlacel 987;

mention may in particular be made of sorbitan oleate, in particular as sold by CRODA under the name Span 80 V;

mention may in particular be made of sorbitan isostearate, in particular as sold by CRODA under the name Arlacel 987;

mention may in particular be made of sorbitan stearate, in particular as sold by COGNIS under the name Dehymuls SMS;

mention may in particular be made of sorbitan laurate, in particular as sold by COGNIS under the name Dehymuls SML;

mention may in particular be made of sorbitan palmitate, in particular as sold by CRODA under the name Span 40;
sucrose esters:

mention may in particular be made of sucrose polystearate, in particular as sold by SISTERNA under the name Sisterna SP10-C;

mention may in particular be made of sucrose distearate, in particular as sold by CRODA under the name Crodesta F-10;
other lipophilic surfactants with an HLB less than or equal to 9 may also be mentioned:

mention may in particular be made of glycol stearate, in particular as sold by CRODA under the name Cithrol EGMS N/E;

mention may in particular be made of PEG-2 stearate, in particular as sold by CRODA under the name Cithrol DEGMS N/E;

mention may in particular be made of methyl glucose isostearate, in particular as sold by EVONIK GOLDSCHMIDT under the name Isolan IS;

mention may in particular be made of cetearyl glucoside (and) cetearyl alcohol, in particular as sold by COGNIS under the name Emulgade PL 68/50.

By way of hydrophilic surfactant in accordance with the present invention, mention is in particular made of sodium lauryl glucose carboxylate (and) lauryl glucoside, in particular as sold by the company Cognis (BASF) under the name Plantapon® LGC Sorb.

By way of lipophilic surfactant in accordance with the present invention, mention is in particular made of polyglyceryl-2 dipolyhydroxystearate, in particular as sold by the company Cognis (BASF) under the name DEHYMULS PGPH.

According to one particular embodiment of the invention, the ratio between the percentage of lipophilic surfactant and the percentage of hydrophilic surfactant, both with respect to active material, is between 7 and 15, in particular between 8 and 12.

According to another particular embodiment, the composition comprises at least sodium lauryl glucose carboxylate (and) lauryl glucoside as hydrophilic surfactant and at least polyglyceryl-2 dipolyhydroxystearate as lipophilic surfactant. The ratio indicated above is also valid in the context of this particular embodiment.

With the proviso of not affecting the phase inversion properties required, the composition according to the present invention may comprise, additionally, one or more surfactants having an HLB between 3 and 15.

Polysaccharide

The composition according to the present invention may also comprise at least one polysaccharide. This polysaccharide may in particular have the effect of providing an increase in consistency of the emulsion.

By way of polysaccharide, mention may in particular be made of scleroglucan gum; xanthan gum and derivatives such as dehydroxanthan; guar gum; tara gum; ghatti gum; sclerotium gum; starches; agar; agarose; carrageenans, such as iota carrageenan, lambda carrageenan and kappa carrageenan; carob flour; alginates; celluloses and derivatives; hydroxypropylguar; pectins and gellan gum.

Among the cellulose derivatives, mention may in particular be made of cellulose esters and/or alkyl ethers, such as ethylcelluloses, propylcelluloses, hydroxyethylcelluloses, hydroxypropylcelluloses or cellulose acetobutyrates.

In the context of the present invention, “sclerotium gum” and “scleroglucan” are equivalent terms, as is Sclerotium rolfsii gum. Indeed, the fungus Sclerotium rolfsii makes it possible to produce scleroglucan.

According to one embodiment, the composition relating to the present invention can contain mixtures of polysaccharides. Among these combinations, mention may in particular be made of:
scleroglucan gum and alginate,
xanthan gum and alginate, and
xanthan gum and guar gum.

The inventors have in fact noted that the consistency of the present composition can be improved by incorporating a first polysaccharide into the initial aqueous phase, preferably under hot conditions, for example at a temperature which may be between 60 and 80° C., then introducing a second polysaccharide at a lower temperature, which may, for example, range from 40 to 45° C., after the formation of the emulsion.

The polysaccharide(s) can be included in the present composition in a content between 0.2% and 3% by weight, preferably between 0.4% and 2.5% by weight, or even from 0.5% to 2% by weight, relative to the total weight of the composition.

Aqueous Phase

The composition according to the invention comprises an aqueous phase comprising water and/or hydrophilic solvents such as polyols.

The amount of water in the composition may range, for example, from 0.5% to 95% by weight, preferably from 1% to 90% by weight, better still from 10% to 80% by weight and even better still from 40% to 75% by weight, relative to the total weight of the composition.

The water used in the composition of the invention may be demineralized pure water, but also mineral water and/or spring water and/or seawater, i.e. the water of the composition may be partially or totally constituted of water chosen from mineral waters, spring waters, seawaters and mixtures thereof. In general, a mineral water is suitable for consumption, which is not always the case with a spring water. Each of these waters contains, inter alia, dissolved minerals and/or trace elements. These waters are known to be employed for specific treatment purposes according to the particular trace elements and minerals that they contain, such as the moisturization and desensitization of the skin or the treatment of certain dermatoses. The terms “mineral waters” and “spring waters” will denote not only natural mineral or spring waters but also natural mineral or spring waters enriched in additional mineral constituents and/or trace elements, and also aqueous mineral solutions and/or solutions containing trace elements prepared from purified water (demineralized or distilled water).

A natural spring water or mineral water used according to the invention may, for example, be chosen from Vittel water, Vichy basin water, Uriage water, Roche Posay water, Bourboule water, Enghien-les-Bains water, Saint Gervais-les-Bains water, Néris-les-Bains water, Allevar-les-Bains water, Digne water, Maizières water, Neyrac-les-Bains water, Lons-le-Saunier water, Eaux Bonnes water, Rochefort water, Saint Christau water, Fumades water, Tercis-les-bains water and Avene water.

The aqueous phase of the composition of the invention may comprise a water-soluble organic solvent chosen, for example, from lower monoalcohols containing from 1 to 8 carbon atoms and in particular 1 to 6 carbon atoms, such as ethanol, isopropanol, propanol, butanol, 1,3-propanediol, polyols, for instance glycerol, propylene glycol, butylene glycol, hexylene glycol, polyethylene glycols such as PEG-8, dipropylene glycol, and mixtures thereof.

According to one preferred embodiment of the invention, the polyol is glycerol which gives better comfort on application. Other polyols can be added to the glycerol, provided that the qualities of the composition are maintained.

The amount of polyol(s) can range, for example, from 0.5% to 15% by weight, preferably from 0.5% to 10% by weight, better still from 1% to 10% by weight, even better still from 2% to 10% by weight and even better still from 2% to 8% by weight, relative to the total weight of the composition.

Fatty Phase

The fatty phase of the composition according to the invention comprises all the liposoluble or lipodispersible compounds present in the composition, including the fatty substances which are liquid at ambient temperature (25° C.) or oils (which form the oily phase), the fatty substances which are solid at ambient temperature, such as waxes, or else pasty compounds, fatty alcohols and fatty acids.

Thus, the composition according to the invention can comprise an oil which can be present in a content ranging from 0.5% to 40% by weight, preferably from 1% to 30% by weight and better still from 5% to 25% by weight, relative to the total weight of the composition.

As oils that may be used in the composition of the invention, examples that may be mentioned include:
hydrocarbon-based oils of animal origin, such as perhydrosqualene;
hydrocarbon-based oils of plant origin, such as liquid triglycerides of fatty acids containing from 4 to 30 carbon atoms, for instance heptanoic or octanoic acid triglycerides, or alternatively, for example, jojoba oil, babassu oil, sunflower oil, olive oil, coconut oil, Brazil nut oil, manila oil, corn oil, soybean oil, marrow oil, grapeseed oil, sesame seed oil, hazelnut oil, apricot oil, macadamia nut oil, arara oil, coriander oil, castor oil, avocado oil, caprylic/capric acid triglycerides, for instance those sold by the company Stearineries Dubois or those sold under the names Miglyol 810, 812 and 818 by the company Dynamit Nobel, and shea butter oil;
synthetic esters and ethers, especially of fatty acids, for instance the oils of formulae R¹COOR² and R¹OR² in which R¹ represents a fatty acid residue or a fatty alcohol residue containing from 8 to 29 carbon atoms and R² represents a branched or unbranched hydrocarbon-based chain containing from 3 to 30 carbon atoms, for instance purcellin oil, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate, triisocetyl citrate, and fatty alcohol heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate;
linear or branched hydrocarbons of mineral or synthetic origin, such as volatile or non-volatile liquid paraffins, and derivatives thereof, petroleum jelly, polydecenes, isohexadecane, isododecane, and hydrogenated polyisobutene such as Parleam® oil;
silicone oils, for instance volatile or non-volatile polymethylsiloxanes (PDMSs) containing a linear or cyclic silicone chain, which are liquid or pasty at ambient temperature, especially volatile silicone oils, in particular cyclopolydimethylsiloxanes (cyclomethicones) such as cyclohexadimethylsiloxane and cyclopentadimethylsiloxane; polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes, 2-phenylethyltrimethylsiloxysilicates and polymethylphenylsiloxanes;
mixtures thereof.

Mention may also be made of the following oils:
esters derived from the reaction of at least one fatty acid comprising at least 6 carbon atoms, preferably from 6 to 26 carbon atoms, better still from 6 to 20 carbon atoms and even better still from 6 to 16 carbon atoms, and of at least one alcohol comprising from 1 to 17 carbon atoms and better still from 3 to 15 carbon atoms; mention may in particular be made of isopropyl myristate, isopropyl palmitate, 2-ethylhexyl caprate/caprylate (or octyl caprate/caprylate), 2-ethylhexyl palmitate, isostearyl neopentanoate, isononyl isononanoate, hexyl laurate, lactic acid esters of fatty alcohols comprising 12 or 13 carbon atoms, and dicaprylyl carbonate, such as the product which is sold under the name CETIOL CC by the company COGNIS,
fatty acid ethers comprising from 6 to 20 carbon atoms, such as dicaprylyl ether (Cetiol OE from Cognis),
glycerol ethers comprising from 6 to 12 carbon atoms, for instance the 2-ethylhexyl ether of glycerol (INCI name: ethylhexylglycerin) such as Sensiva SC 50 from the company Schulke & Mayr GmbH.

Preferably, the oily phase of the composition (comprising the oils) is composed of a mixture of plant oils, of alkanes, or else of esters.

The cosmetic compositions of the invention may also contain adjuvants that are common in the cosmetics field, such as antioxidants, preservatives, fragrances, fragrance peptizers, colorants, fillers, or hydrophilic or lipophilic active agents. The nature of the adjuvants and the amounts thereof should be such that they do not modify the properties of the composition according to the invention. The amounts of these adjuvants are those conventionally used in the cosmetics field, for example from 0.001% to 10% of the total weight of the composition.

As active agents that may be used in the composition of the invention, examples that may be mentioned include calmatives such as allantoin and bisabolol; floral waters such as linden tree water and cornflower water; glycyrrhetinic acid and salts thereof; antibacterial agents such as octopirox, triclosan and triclocarban; essential oils; vitamins, for instance retinol (vitamin A), ascorbic acid (vitamin C), tocopherol (vitamin E), niacinamide (vitamin PP or B3), panthenol (vitamin B5) and derivatives thereof, for instance esters of these vitamins (palmitate, acetate, propionate), magnesium ascorbyl phosphate, glycosyl vitamin C or glucopyranosyl ascorbic acid (ascorbyl glucoside); coenzymes such as coenzyme Q10 or ubiquinone and coenzyme R or biotin; protein hydrolyzates; plant extracts and especially plankton extracts; and mixtures thereof.

Needless to say, a person skilled in the art will take care to select the optional additive(s) to be added to the composition according to the invention such that the advantageous properties intrinsically associated with the composition in accordance with the invention are not, or are not substantially, adversely affected by the envisaged addition.

Fillers that may be mentioned include mineral fillers such as talc or magnesium silicate (particle size: 5 microns) sold under the name LUZENAC 15 MOO® by the company LUZENAC, kaolin or aluminum silicate, for instance the product sold under the name KAOLIN SUPREME® by the company IMERYS, or organic fillers such as starch, for instance the product sold under the name AMIDON DE MAIS B® by the company ROQUETTE, Nylon microspheres such as those sold under the name ORGASOL 2002 UD NAT COS® by the company ATOCHEM, microspheres based on expanded vinylidene chloride/acrylonitrile/methacrylonitrile copolymer containing isobutane, such as the products sold under the name EXPANCEL 551 DE® by the company EXPANCEL. Fibers, for instance nylon fibers (POLYAMIDE 0.9 DTEX 0.3 MM sold by Etablissements PAUL BONTE), or cellulose or <<Rayon>> fibers (RAYON FLOCK RCISE NOOO3 MO4® sold by the company CLAREMONT FLOCK CORPORATION), may also be added to the composition of the invention. The cellulose fibres under the trade name RCIBE N0003 02M sold by the company Claremont Flock may be added. The cellulose beads under the trade name Cellulobeads by the company Daito Kasei may also be added.

The compositions according to the invention are intended to be applied to keratin materials such as the skin (body, face, eyes, scalp) and can constitute in particular tinted care or makeup products for keratin materials.

The composition according to the invention may also comprise nacres. The term “nacres” should be understood as meaning iridescent or noniridescent colored particles of any form, especially produced by certain molluscs in their shell or alternatively synthesized, which have a color effect via optical interference.

A composition according to the invention may comprise from 1% to 80% by weight, preferably from 5% to 60% by weight and better still from 10% to 40% by weight of nacres, relative to the total weight of said composition.

The nacres may be chosen from nacreous pigments such as titanium mica coated with an iron oxide, titanium mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic colorants.

Examples of nacres that may also be mentioned include natural mica coated with titanium oxide, with iron oxide, with natural pigment, boron nitride or with bismuth oxychloride.

Among the commercially available nacres that may be mentioned are the nacres TIMICA, FLAMENCO and DUOCHROME (on mica base) sold by the company ENGELHARD, the TIMIRON nacres sold by the company MERCK, the PRESTIGE nacres on mica base sold by the company ECKART and the SUNSHINE nacres on synthetic mica base sold by the company SUN CHEMICAL.

Mention may also be made of the boron nitride under the trade name SOFTOUCH BORON NITRIDE POWDER CC6058 sold by the company Momentive Performance materials.

The nacres may more particularly have a yellow, pink, red, bronze, orangey, brown, gold and/or coppery color or glint.

As illustrations of nacres that may be used in the context of the present invention, mention may in particular be made of gold-colored nacres sold especially by the company ENGELHARD under the name Brilliant gold 212G (Timica), Gold 222C (Cloisonne), Sparkle gold (Timica), Gold 4504 (Chromalite) and Monarch gold 233X (Cloisonne); the bronze nacres sold especially by the company MERCK under the names Bronze fine (17384) (Colorona) and Bronze (17353) (Colorona) and by the company ENGELHARD under the name Super bronze (Cloisonne); the orange nacres sold especially by the company ENGELHARD under the names Orange 363C (Cloisonne) and Orange MCR 101 (Cosmica) and by the company MERCK under the names Passion orange (Colorona) and Matte orange (17449) (Microna); the brown-tinted nacres sold especially by the company ENGELHARD under the names Nu-antique copper 340XB (Cloisonne) and Brown CL4509 (Chromalite); the nacres with a copper glint sold especially by the company ENGELHARD under the name Copper 340A (Timica); the nacres with a red glint sold especially by the company MERCK under the name Sienna fine (17386) (Colorona); the nacres with a yellow glint sold especially by the company ENGELHARD under the name Yellow (4502) (Chromalite); the red-tinted nacres with a golden glint sold especially by the company ENGELHARD under the name Sunstone G012 (Gemtone); the pink nacres sold especially by the company ENGELHARD under the name Tan opale G005 (Gemtone); the black nacres with a golden glint sold especially by the company ENGELHARD under the name Nu antique bronze 240 AB (Timica); the blue nacres sold especially by the company MERCK under the name Matte blue (17433) (Microna); the white nacres with a silvery glint sold especially by the company MERCK under the name Xirona Silver; and the golden-green pinkish-orange nacres sold especially by the company MERCK under the name Indian summer (Xirona), and mixtures thereof.

Advantageously, the nacres in accordance with the invention are micas coated with titanium dioxide or with iron oxide, and also bismuth oxychloride.

The composition according to the present invention can also comprise particles with metallic glints. For the purposes of the present invention, the term “particles with a metallic glint” means any compound whose nature, size, structure and surface finish allow it to reflect the incident light, especially in a noniridescent manner.

A composition according to the invention may comprise from 1% to 50% by weight and preferably from 1% to 20% by weight of particles with a metallic glint, relative to the total weight of said composition.

Particles with a substantially flat outer surface are also suitable, since they can, if their size, structure and surface finish allow it, more easily give rise to a strong specular reflection, which may then be termed a mirror effect.

The particles with a metallic glint that may be used in the invention may, for example, reflect light in all the components of the visible region without significantly absorbing one or more wavelengths. The spectral reflectance of these particles may, for example, be greater than 70% and better still at least 80%, or even 90% or 95%, in the range 400-700 nm.

These particles generally have a thickness of less than or equal to 1 μm, especially less than or equal to 0.7 μm and in particular less than or equal to 0.5 μm.

The particles with a metallic glint that may be used in the invention are in particular chosen from:
particles of at least one metal and/or of at least one metal derivative;
particles comprising a single-material or multi-material organic or inorganic substrate, at least partially coated with at least one layer with a metallic glint comprising at least one metal and/or at least one metal derivative; and
mixtures of said particles.

Among the metals that may be present in said particles, mention may be made, for example, of Ag, Au, Cu, Al, Ni, Sn, Mg, Cr, Mo, Ti, Zr, Pt, Va, Rb, W, Zn, Ge, Te and Se, and mixtures or alloys thereof. Ag, Au, Cu, Al, Zn, Ni, Mo and Cr and mixtures or alloys thereof (for example bronzes and brasses) are preferred metals.

The term “metal derivatives” denotes compounds derived from metals, especially oxides, fluorides, chlorides and sulfides.

Among the metal derivatives that may be present in said particles, mention may be made especially of metal oxides, for instance titanium oxide, especially TiO₂, iron oxide, especially Fe₂O₃, tin oxide, chromium oxide, barium sulfate and the following compounds: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂, and mixtures or alloys thereof.

Illustrations of these particles that may be mentioned include aluminum particles, such as those sold under the names STARBRITE 1200 EAC® by the company SIBERLINE and METALURE® by the company ECKART.

Mention may also be made of metal powders of copper or of alloy mixtures such as the references 2844 sold by the company RADIUM BRONZE, metallic pigments, for instance aluminum or bronze, such as those sold under the names ROTOSAFE 700 from the company ECKART, silica-coated aluminum particles sold under the name VISIONAIRE BRIGHT SILVER from the company ECKART, and metal alloy particles, for instance the silica-coated bronze (alloy of copper and zinc) powders sold under the name Visionaire Bright Natural Gold from the company Eckart.

As illustrations of particles of this second type, mention may be made more particularly of:

Glass particles coated with a metallic layer, especially those described in documents JP-A-09188830, JP-A-10158450, JP-A-10158541, JP-A-07258460 and JP-A-05017710.

As illustrations of these particles comprising a glass substrate, mention may be made of those coated, respectively, with silver, gold or titanium, in the form of platelets, sold by the company NIPPON SHEET GLASS under the name MICROGLASS METASHINE. Particles with a silver-coated glass substrate, in the form of platelets, are sold under the name MICROGLASS METASHINE REFSX 2025 PS by the company TOYAL. Particles with a glass substrate coated with nickel/chromium/molybdenum alloy are sold under the name CRYSTAL STAR GF 550 and GF 2525 by this same company. Those coated either with brown iron oxide or with titanium oxide, tin oxide or an oxide of a mixture thereof, for instance those sold under the name REFLECKS by the company ENGELHARD or those sold under the reference METASHINE MC 2080GP by the company NIPPON SHEET GLASS.

These metal-coated glass particles may be coated with silica, for instance those sold under the name METASHINE series PSS1 or GPS 1 by the company NIPPON SHEET GLASS;

Particles comprising a spherical glass substrate optionally coated with a metal, especially those sold under the name PRIZMALITE MICROSPHERE by the company PRIZMALITE INDUSTRIES.

Pigments of the METASHINE 1080R range sold by the company NIPPON SHEET GLASS CO. LTD are also suitable for the invention. These pigments, more particularly described in patent application JP 2001-11340, are C-GLASS glass flakes comprising 65% to 72% SiO₂, coated with a layer of titanium oxide of rutile type (TiO₂). These glass flakes have a mean thickness of 1 micron and a mean size of 80 microns, i.e. a mean size/mean thickness ratio of 80. They have blue, green or yellow glints or a silver tint depending on the thickness of the TiO₂ layer;

Particles comprising a silver-coated borosilicate substrate, also known as “white nacres”.

Particles comprising a metal substrate such as aluminum, copper or bronze, in the form of platelets, are sold under the trade name STARBRITE by the company SILBERLINE and under the name VISIONAIRE by the company ECKART;

Particles comprising a synthetic mica substrate coated with titanium dioxide, and for example particles with a size of between 80 and 100 μm, comprising a synthetic mica (fluorophlogopite) substrate coated with titanium dioxide representing 12% of the total weight of the particle, sold under the name PROMINENCE by the company NIHON KOKEN.

The particles with a metallic glint may also be chosen from particles formed from a stack of at least two layers with different refractive indices. These layers may be of polymeric or metallic nature and may especially include at least one polymer layer.

Thus, the particles with a metallic effect may be particles derived from a multilayer polymer film.

The choice of materials intended to constitute the various layers of the multilayer structure is obviously made so as to give the particles thus formed the desired metallic effect.

Such particles are especially described in WO 99/36477, U.S. Pat. No. 6,299,979 and U.S. Pat. No. 6,387,498 and more particularly identified below in the goniochromatic section.

Advantageously, the particles with a metallic glint in accordance with the invention are particles with a spherical or non-spherical glass substrate, and also particles with a metallic substrate.

Some of these materials may also be used in a composite form. Composite pigments that may especially be mentioned in this respect include those described in patent EP 1 184 426. These composite pigments may especially be composed of particles comprising a inorganic core at least partially covered with an organic pigment and at least one binder for attaching the organic pigments to the core.

The examples that follow are given as illustrations of the invention and are not limiting in nature. All the amounts are given as weight percentages relative to the total weight of the composition. The names of the compounds are indicated, depending on the case, as the chemical names or the INCI names.

### COMPOSITION EXAMPLES

### Example 1

### Enhancing Care Product

| | | % by weight relative to the |
|---|---|---|
| | | total weight of the |
| Phases | Composition | composition |
| A1 | Water | 59.2 |
| A1 | Benzyl alcohol | 0.5 |
| A1 | Dehydroxanthan | 0.8 |
| A2 | Sodium lauryl glucose | 1.5 |
| | carboxylate (and) lauryl | |
| | glucoside^{(a)} | |
| B | Macadamia nut oil | 7.5 |
| B | Dicaprylyl carbonate | 7.5 |
| B | Polyglyceryl-2^{(b)} | 4 |
| | dipolyhydroxystearate | |
| C | Dicaprylyl ether | 4.997 |
| C | Iron oxides (and) Disodium | 3.496 |
| | stearoyl glutamate (and) | |
| | Aluminum hydroxide⁽¹⁾ | |
| C | Titanium dioxide (and) | 10.507 |
| | Disodium stearoyl glutamate | |
| | (and) Aluminum hydroxide⁽²⁾ | |

^{(a)}Sold by Cognis (BASF) under the name Plantapon LG Sorb
^{(b)}Sold by Cognis (BASF) under the name Dehymuls DPGP
⁽¹⁾and ⁽²⁾Sold by Miyoshi Kasei. These particles have an average size of greater than 450 nanometers. Thus, the particles (1) have an average size equal to 4.435 micrometers and (2) have an average size equal to 0.495 micrometers.

Method for Preparing and Evaluating the Composition

Phase A1 is first of all prepared by swelling dehydrated xanthan gum at 75° C. in water. Phase A2 is then introduced into phase A1, and then phase B is heated to 75° C.

Phase B is then poured into phase A at 75° C. with stirring so as to form an emulsion, then phase C is incorporated into the mixture at ambient temperature.

This composition was subsequently evaluated by an esthetician with a fluid foundation finger action on a panel of 6 models having various types of skin, the models being between 20 and 55 years old. A fresh sensation on application was demonstrated.

### Example 2

### Enhancing Care Product

| | | % by weight relative |
|---|---|---|
| | | to the total weight of |
| Phases | Composition | the composition |
| A1 | Water | 69 |
| A1 | Benzyl alcohol | 0.5 |
| A1 | Xanthan gum | 0.67 |
| A2 | Sodium lauryl glucose | 1.5 |
| | carboxylate (and) lauryl | |
| | glucoside^{(a)} | |
| B1 | Macadamia nut oil | 7.5 |
| B1 | Dicaprylyl carbonate | 7.5 |
| B1 | Polyglyceryl-2 | 4 |
| | dipolyhydroxystearate^{(b)} | |
| B2 | Dicaprylyl ether | 1.578 |
| B2 | Iron oxides (and) Disodium | 1.104 |
| | stearoyl glutamate (and) | |
| | Aluminum hydroxide⁽¹⁾ | |
| B2 | Titanium dioxide (and) | 3.318 |
| | Disodium stearoyl glutamate | |
| | (and) Aluminum hydroxide⁽²⁾ | |
| C | Guar gum | 0.33 |
| C | Glycerol | 3 |

^{(a)}Sold by Cognis (BASF) under the name Plantapon LG Sorb
^{(b)}Sold by Cognis (BASF) under the name Dehymuls DPGP
⁽¹⁾and ⁽²⁾Sold by Miyoshi Kasei. These particles have an average size of greater than 450 nanometers. Thus, the particles (1) have an average size equal to 4.435 micrometers and (2) have an average size equal to 0.495 micrometers.

Method for Preparing the Composition

Phase A1 is first of all prepared by swelling the xanthan gum at 80° C. in water, then phase A2 is introduced into phase A1.

Phase B1 is then heated to 80° C., then phase B2 is introduced into phase B1.

Phase B is then poured into phase A at 60° C. with stirring so as to form an emulsion, then phase C is incorporated, before allowing the resulting composition to cool, with slow stirring, to ambient temperature.

### Example 3

### Enhancing Care Product

| | | % by weight relative to |
|---|---|---|
| | | the total weight of the |
| Phases | Composition | composition |
| A1 | Water | 66.983 |
| A1 | Calcium chloride | 0.017 |
| A1 | Benzyl alcohol | 0.5 |
| A1 | Sodium alginate | 0.33 |
| A2 | Sodium lauryl glucose | 1.5 |
| | carboxylate (and) lauryl | |
| | glucoside^{(a)} | |
| B1 | Macadamia nut oil | 7.5 |
| B1 | Dicaprylyl carbonate | 7.5 |
| B1 | Polyglyceryl-2 | 4 |
| | dipolyhydroxystearate^{(b)} | |
| B2 | Dicaprylyl ether | 1.578 |
| B2 | Iron oxides (and) | 1.104 |
| | Disodium stearoyl | |
| | glutamate (and) | |
| | Aluminum hydroxide⁽¹⁾ | |
| B2 | Titanium dioxide (and) | 3.318 |
| | Disodium stearoyl | |
| | glutamate (and) | |
| | Aluminum hydroxide⁽²⁾ | |
| C | Xanthan gum | 0.67 |
| D | Alcohol | 5 |

^{(a)}Sold by Cognis (BASF) under the name Plantapon LG Sorb
^{(b)}Sold by Cognis (BASF) under the name Dehymuls DPGP
⁽¹⁾and ⁽²⁾Sold by Miyoshi Kasei. These particles have an average size of greater than 450 nanometers. Thus, the particles (1) have an average size equal to 4.435 micrometers and (2) have an average size equal to 0.495 micrometers.

Method for Preparing the Composition

Phase A1 is first of all prepared by swelling the alginate gel at 75° C. in water, then phase A2 is introduced into phase A1.

Phase B1 is then heated to 55° C., then phase B2 is introduced into phase B1.

Phase B is then poured into phase A at 55° C. with stirring so as to form an emulsion, then phase C is incorporated as a fine rain, with stirring, before allowing the resulting composition to cool, with stirring, to ambient temperature. Phase D is then in turn incorporated into the composition at ambient temperature.

### Example 4

### Enhancing Care Product

| | | % by weight relative to the |
|---|---|---|
| | | total weight of the |
| Phases | Composition | composition |
| A1 | Water | 66.983 |
| A1 | Calcium chloride | 0.017 |
| A1 | Benzyl alcohol | 0.5 |
| A1 | Sodium alginate | 0.33 |
| A2 | Sodium lauryl glucose | 1.5 |
| | carboxylate (and) lauryl | |
| | glucoside^{(a)} | |
| B1 | Jojoba seed oil | 5 |
| B1 | Dicaprylyl ether | 5 |
| B1 | Undecane (and) Tridecane | 5 |
| B1 | Polyglyceryl-2 | 4 |
| | dipolyhydroxystearate^{(b)} | |
| B2 | Dicaprylyl ether | 1.578 |
| B2 | Iron oxides (and) Disodium | 1.104 |
| | stearoyl glutamate (and) | |
| | Aluminum hydroxide⁽¹⁾ | |
| B2 | Titanium dioxide (and) | 3.318 |
| | Disodium stearoyl glutamate | |
| | (and) Aluminum hydroxide⁽²⁾ | |
| C | Sclerotium rolfssii gum | 0.67 |
| D | Alcohol | 5 |

^{(a)}Sold by Cognis (BASF) under the name Plantapon LG Sorb
^{(b)}Sold by Cognis (BASF) under the name Dehymuls DPGP
⁽¹⁾and ⁽²⁾Sold by Miyoshi Kasei. These particles have an average size of greater than 450 nanometers. Thus, the particles (1) have an average size equal to 4.435 micrometers and (2) have an average size equal to 0.495 micrometers.

Method for Preparing and Evaluating the Composition

Phase A1 is first of all prepared by swelling the alginate gel at 80° C. in water, then phase A2 is introduced into phase A1.

Phase B1 is then heated to 35° C., then phase B2 is introduced into phase B1.

Phase B is then poured into phase A at 35° C. with stirring so as to form an emulsion, then phase C is incorporated, with stirring, before allowing the resulting composition to cool, with stirring, to ambient temperature. Phase D is then in turn incorporated into the composition at ambient temperature.

This composition was then evaluated by an esthetician according to the care product hand action on a panel of 6 caucasian women, including: 1 normal to dry skin, 3 normal skin, 1 mixed to oily skin and 1 oily skin.

By virtue of its beige film, an esthetic skin finish was demonstrated, and also a virtually systematic reduction in dyschromia, and a fresh sensation felt by the panelists.

### Example 5

### Foundation

| | | % by weight relative to |
|---|---|---|
| | | the total weight of the |
| Phases | Composition | composition |
| A1 | Water | 44.5 |
| A1 | Glycerol | 5 |
| A1 | Propanediol | 5 |
| A1 | Benzyl alcohol | 0.5 |
| A1 | Sodium alginate | 0.1 |
| A2 | Xanthan gum | 0.4 |
| B1 | Sodium lauryl glucose carboxylate | 1.5 |
| | (and) lauryl glucoside^{(a)} | |
| B1 | Jojoba seed oil | 5 |
| B1 | Dicaprylyl ether | 9.734 |
| B1 | Undecane (and) Tridecane | 5 |
| B1 | Polyglyceryl-2 | 4 |
| | dipolyhydroxystearate^{(b)} | |
| B2 | Iron oxides (and) Disodium | 3.312 |
| | stearoyl glutamate (and) | |
| | Aluminum hydroxide⁽¹⁾ | |
| B2 | Titanium dioxide (and) Disodium | 9.954 |
| | stearoyl glutamate (and) | |
| | Aluminum hydroxide⁽²⁾ | |
| C | Sclerotium rolfssii gum | 0.4 |
| D | Alcohol | 5 |
| E | 10% aqueous solution of citric acid | 0.5 |
| F | Fragrance | 0.1 |

^{(a)}Sold by Cognis (BASF) under the name Plantapon LG Sorb
^{(b)}Sold by Cognis (BASF) under the name Dehymuls DPGP
⁽¹⁾and ⁽²⁾Sold by Miyoshi Kasei. These particles have an average size of greater than 450 nanometers. Thus, the particles (1) have an average size equal to 4.435 micrometers and (2) have an average size equal to 0.495 micrometers.

Method for Preparing the Composition

Phase A1 is first of all prepared by swelling the alginate at 80° C. in water, then phase A2 (xanthan gum) is introduced into phase A1.

Phase B1 is then heated to 55° C., then phase B2 is introduced into phase B1.

Phase B is then poured into phase A at 55° C. with stirring so as to form an emulsion, then phase C is incorporated, with stirring, before allowing the resulting composition to cool, with stirring, to ambient temperature. Phases D, E and F are then in turn incorporated into the composition at ambient temperature.

All the compositions exemplified exhibit good cosmetic properties, in particular both a fresh sensation on application and appropriate coverage.

### Example 6

### Compositions with Various Polysaccharides

Six compositions (6.1 to 6.6) were prepared with various polysaccharides.

| | 6.1 | 6.2 | 6.3 | 6.4 | 6.5 | 6.6 |
|---|---|---|---|---|---|---|
| Dehydroxanthan* | 0.80 | | | | | 0.67 |
| Carob flour* | | 0.60 | | | | |
| Xanthan gum*$ | | 0.4 | 0.67 | 0.67 | | |
| Guar gum$ | | | 0.33 | | | 0.33 |
| Alginate* | | | | 0.33 | 0.33 | |
| Calcium chloride | | | | 0.02 | 0.02 | |
| Sclerotium gum$ | | | | | 0.67 | |
| Hydrophilic SA | 1.5 | 1.5 | 1.5 | 1.5 | 1.5 | 1.5 |
| Lipophilic SA | 4 | 4 | 4 | 4 | 4 | 4 |
| Dicaprylyl | 7.5 | 7.5 | 7.5 | 7.5 | 7.5 | 7.5 |
| carbonate | | | | | | |
| Macadamia oil | 7.5 | 7.5 | 7.5 | 7.5 | 7.5 | 7.5 |
| Ethanol | | | | 5.0 | 5.0 | 5.0 |
| Preservative | 0.5 | 0.5 | 0.5 | 0.5 | 0.5 | 0.5 |
| Dicaprylyl ether | 1.6 | 1.6 | 1.6 | 1.6 | 1.6 | 1.6 |
| Pigments | 4.4 | 4.4 | 4.4 | 4.4 | 4.4 | 4.4 |

The lipophilic surfactant “lipophilic SA” is polyglyceryl-2 dipolyhydroxystearate.

The hydrophilic surfactant “hydrophilic SA” is sodium lauryl glucose carboxylate (and) lauryl glucoside.

Demineralized water makes the compositions up to 100%.

When the composition comprises two polysaccharides, the first is incorporated into the aqueous phase before the emulsion and the second is added by “sprinkling” it after emulsion.

The aqueous phase is gelled before emulsification, otherwise phase inversion occurs during the W/O emulsion production.

The gelling agents which can be the first ones are marked with an * in the table.

The gelling agents which can be the second ones are marked with a $ in the table.

### CONCLUSION

Phase inversion was observed during the application of the compositions to the skin. The desired fresh sensory effect was, moreover, observed while at the same time providing good coverage.

1. A cosmetic composition for caring for and/or making up keratin materials which is in the form of an oil-in-water emulsion which undergoes phase inversion when it is applied to said keratin materials, comprising:
(i) at least one pigment having a particle size greater than 100 nanometers, in particular greater than 300 nanometers, or even than 400 nanometers, and
(ii) a combination of at least one hydrophilic surfactant and at least one lipophilic surfactant.

2. The cosmetic composition as claimed in claim 1, in which the pigment is chosen from titanium oxide, titanium dioxide, zirconium oxide, zirconium dioxide, cerium oxide or cerium dioxide and also zinc oxide, iron oxide or chromium oxide, ferric blue, manganese violet, ultramarine blue and chromium hydrate, and mixtures thereof.

3. The cosmetic composition as claimed in claim 1, in which the hydrophilic surfactant is chosen from a surfactant having an HLB greater than or equal to 13 which can be chosen from alkyl mono- and polyglucosides, anionic derivatives of alkyl polyglucosides, fatty acid esters, in particular of glycerol, polysorbates and sucrose esters or else chosen from laureth-4 phosphate, lauroyl sarcosine and glycereth-25 PCA isostearate.

4. The cosmetic composition as claimed in claim 1, in which the lipophilic surfactant is chosen from a surfactant having an HLB less than or equal to 9 which can be chosen from fatty acid esters, in particular of glycerol and of polyglycerol, polysorbates and sucrose esters or else chosen from glycol stearate, PEG-2 stearate, methyl glucose isostearate, sorbitan palmitate, and cetearyl glucoside (and) cetearyl alcohol.

5. The cosmetic composition as claimed in claim 1, in which the hydrophilic surfactant comprises at least one alkyl monoglycoside, such as an alkyl monoglucose or alkyl monoglucoside and an anionic derivative of an alkyl monoglucoside such as an alkyl monoglucoside carboxylate, which is in particular carboxymethylated.

6. The cosmetic composition as claimed in claim 1, in which the hydrophilic surfactant is sodium lauryl glucose carboxylate (and) lauryl glucoside and the lipophilic surfactant is polyglyceryl-2 dipolyhydroxystearate.

7. The cosmetic composition as claimed in claim 1, in which the composition also comprises at least one polysaccharide.

8. The composition as claimed in claim 1, in which the polysaccharide is chosen from scleroglucan gum; xanthan gum and derivatives thereof such as dehydroxanthan; guar gum; tara gum; ghatti gum; sclerotium gum; starches; agar; agarose; carrageenans, such as iota carrageenan, lambda carrageenan and kappa carrageenan; carob flour; alginates; celluloses and derivatives thereof; hydroxypropylguar; pectins and gellan gum.

9. The cosmetic composition as claimed in claim 1, in which the polysaccharide is chosen from a combination:
scleroglucan gum and alginate,
xanthan gum and alginate, and
xanthan gum and guar gum.

10. The cosmetic composition as claimed in claim 1, in which the content of polysaccharide(s) is between 0.2% and 3% by weight, preferably between 0.4% and 2.5% by weight, or even from 0.5% to 2% by weight, relative to the total weight of the composition.

11. The cosmetic composition as claimed in claim 1, characterized in that it contains a fatty phase which includes one or more oils, in particular plant oils, linear oils or else esters.

12. The cosmetic composition as claimed in claim 1, characterized in that it also comprises one or more active agents, one or more fillers and/or one or more nacres.

13. A cosmetic treatment process for keratin materials, characterized in that a cosmetic composition as claimed in claim 1 is applied to said keratin materials.
